(19) United States
(12) Patent Application Publication
(10) Pub. No.: US 2012/0190911 A1
(43) Pub. Date: Jul. 26, 2012

(54) LOW TEMPERATURE HYPERTHERMIA SYSTEM FOR THERAPEUTIC TREATMENT OF INVASIVE AGENTS

(75) Inventors: Daniel B. McKenna, Vail, CA (US); Robert J. Tondu, Houston, TX (US)

(73) Assignee: Actium BioSystems, LLC, Boulder, CO (US)

(21) Appl. No.: 13/012,572

(22) Filed: Jan. 24, 2011

(51) Int. Cl. A61M 37/00; A61N 2/00; A61N 1/00

(52) U.S. Cl. 600/10; 604/20; 604/21

(57) ABSTRACT

The Low Temperature Hyperthermia System illuminates nano-particles, which are implanted in a living organism at the locus of the cancer or into the cancer cells, with a precisely determined energy field. This energy field ensures that the optimal cancer cell and cancer stem cell destruction temperature of 42° C. is not exceeded in the tissue, which minimizes the release of Heat Shock Proteins and cancer stem cells. The Low Temperature Hyperthermia System uses specially designed nano-particles that exhibit a specific temperature rise in a given illumination energy field and then have no further temperature rise even if the applied illumination energy field increases beyond the optimal level. Alternatively, the nano-particles exhibit a tightly controlled temperature rise based on a pre-determined illumination energy field strength. This innovative approach can also use radiation and/or chemotherapy in conjunction with the nano-particle illumination to kill the majority of the cancer cells.

### CROSS-REFERENCE TO RELATED APPLICATIONS

This application is related to U.S. patent applications titled “System For Correlating Energy Field Characteristics With Target Particle Characteristics In The Application Of An Energy Field To A Living Organism For Treatment Of Invasive Agents”; “System For Correlating Energy Field Characteristics With Target Particle Characteristics In The Application Of An Energy Field To A Living Organism For Detection Of Invasive Agents”; “System For Correlating Energy Field Characteristics With Target Particle Characteristics In The Application Of An Energy Field To A Living Organism For Imaging and Treatment Of Invasive Agents”; “System For Automatically Amending Energy Field Characteristics In The Application Of An Energy Field To A Living Organism For Treatment Of Invasive Agents”, and “System For Defining Energy Field Characteristics To Illuminate Nano-Particles Used To Treat Invasive Agents,” all filed on the same date as the present application.

### FIELD OF THE INVENTION

This invention relates to the field of destruction of invasive agents, such as pathogens and cancers, which are located in living organisms and, more particularly, to a system that matches input energy field characteristics, as applied to the living organism, with the characteristics of particles which are infused into the living tissue.

### BACKGROUND OF THE INVENTION

It is a problem in the field of cancer treatment that a non-terminal attack on cancer cells can cause the cancer to rebound at an even higher rate than the initial infection, due to the propagation of cancer stem cells or the release of other cells during the cancer treatment. This process of cancer cell metastasis from a primary site to a secondary site is particularly prevalent in cancers such as triple negative breast cancer. Research indicates that cancer cells emit “Heat Shock Proteins” that tell the cancer it is under attack and that the cancer should respond by emitting cancer stem cells or other cancer survival cells, to build a cancer infection in one or more new locations in the living organism. Intracellular Heat Shock Proteins are highly expressed in cancer cells and are essential to the survival of these cell types. These Heat Shock Proteins enable the cancer cell to survive and recover from stressful conditions by as yet incompletely understood mechanisms.

Thus, poorly regulated heat-based cancer treatment methods, such as microwave hyperthermia, can have the unintended effects of partially killing the cancer and stimulating the production of Heat Shock Proteins and cancer stem cells, thereby ensuring that the cancer survives at its present site and spreads to new locations in the living organism. These poorly regulated heat-based cancer treatment methods typically cause a large temperature variance across a tumor, which is undesirable for the reasons noted above. In addition, non-selective microwave hyperthermia heats healthy tissue along with cancerous tissue without any temperature discrimination, which can harm healthy tissue in the process. Thus, killing cancer cells with microwave-based hyperthermia is not the optimal approach to cancer treatment and can have negative consequences to the living organism.

Other cancer treatment regimens, such as chemotherapy and radiation, can also cause the creation and release of Heat Shock Proteins, sometimes called “Stress Proteins.” Any time these proteins are released, they signal that the cancer is seeking methods to survive. Heat Shock Proteins may be active in the development of resistance to both stressful conditions and anti-cancer agents, including cytotoxic drugs. Thus, it is desirable to find a method to treat cancer and minimize the release of Heat Shock Proteins.

Since one objective of cancer treatment is to minimize the release and propagation of cancer stem cells, it is also desirable to change the biological environment to negatively impact cancer stem cells. Cancer stem cells prefer a low oxygen or hypoxic environment; therefore, it is desirable to increase oxygen levels to those regions inhabited by cancer stem cells. Low Temperature Hypothermia does just this. It improves re-oxygenation and cell respiration, further stressing the cancer stem cells, thereby increasing cancer cell and cancer stem cell death rates. In contrast, high temperature cancer cell destruction does not realize these biological benefits.

An improvement to the current cancer treatment protocols includes cancer cell targeting by the use of energy-absorbing nano-particles to optimize a temperature differential between cancerous and healthy tissue. While this minimizes the heat damage to healthy tissue or cells, this approach can still have “misses,” since the probability that every cancer cell has been destroyed is not 100%. These “misses” are heat stressed cancer cells which further emit cancer stem cells/other cells to propagate and re-grow new cancer cells in different locations of the body.

A cancer treatment protocol which overcomes these limitations (only for cancers which are very near the surface of the skin) distributes gold nano-shells in vivo to cancer cells and then treats the cancer with radiation, where the nano-particles do not enhance or impair the radiation treatment. The nano-particles are given in advance of the radiation treatment to ensure that the nano-particles are on site for the next treatment, which uses lasers to illuminate and heat the gold nano-shells to 42° C. This temperature is not harmful to healthy tissue, but the 42° C. destroys the radiation-stressed cancer cells; and these low temperature-stressed cancer cells emit lower Heat Shock Protein levels and do not release cancer stem cells/other cells. However, this approach can only treat cancers which are at or near the surface of the skin, since the laser illumination cannot penetrate very deep beyond the surface of the skin.

What is needed is a cancer treatment that is universal and independent of where the tumor or cancerous region is located, where tight temperature control is realized in the tumor or, better yet, the cancer cell itself.

### BRIEF SUMMARY OF THE INVENTION

The present Low Temperature Hyperthermia System For Therapeutic Treatment Of Invasive Agents (termed “Low Temperature Hyperthermia System” herein) differentiates between cancerous and healthy tissue and provides a means to ensure that heat stressed cancer cells do not emit cancer stem cells or Heat Shock Proteins. The Low Temperature Hyperthermia System illuminates nano-particles, which are implanted in a living organism at the locus of the cancer or into the cancer cells, with a precisely determined energy field. This energy field ensures that the optimal cancer cell and cancer stem cell destruction temperature of 42° C. is not exceeded in the tissue, which minimizes the release of Heat Shock Proteins and cancer stem cells. The Low Temperature Hyperthermia System uses specially designed nano-particles that exhibit a specific temperature rise in a given energy field and then have no further temperature rise even if the applied energy field increases beyond the optimal level. Alternatively, the nano-particles exhibit a tightly controlled temperature rise based on pre-determined energy field strength. The energy field that is applied is either an electric field (E-Field) or a magnetic field (H-Field) or a combination of both, as an E- and H-Field, or via an orthogonal field such as an EM-Field. This ensures that an optimal temperature, which for the purpose of this description is selected to be 42° C., is not exceeded in the tissue to minimize the release of Heat Shock Proteins while further stressing the cancer cells so that they die, versus emitting cancer stem cells/other cells. It also ensures that healthy tissue is not harmed, should errant nano-particles end up in healthy tissue.

This Low Temperature Hyperthermia System can pre-treat the cancerous site with radiation or chemotherapy to kill the majority of the cancer cells, followed by the application of E-Field or H-Field or EM-Field radiation to the nano-particles to realize a temperature rise from the ambient temperature to 42° C. in the cancer cells. The advantages realized by this treatment protocol are significant: virtually any tumor location can be treated, the release of Heat Shock Proteins is minimized (at 42° C.), errant nano-particles in a healthy cell do not harm a healthy cell at 42° C., cancer cells are kept at a nominal 42° C. (or some other optimum temperature) to ensure that the already stressed cancer cells (from radiation or chemotherapy) are continuing to die, and cancer stem cells are not released.

In addition, maintaining a temperature of 42° C. in the tissue causes other biological benefits: re-oxygenation, apoptosis and respiration inhibition, increased vessel pore size, and increased perfusion. Of these, re-oxygenation is very important, since cancer stem cells prefer to live in a hypoxic environment. Increasing the level of oxygen in and around cancer stem cells is a significant method to further stress and kill cancer stem cells.

Separately, a third killing element can be added—if the nano-particle is a temperature sensitive liposome, the liposome shell will “melt” at a design temp which is less than 42° C., wherein a cytotoxin can be released. This third killing method, the released cytotoxin, can be part of a multi-pronged approach to kill deep seated cancer tumors.

The Low Temperature Hyperthermia System realizes many advantages over the existing art:
It is no longer necessary to pre-image to ensure the nano-particles are in the correct location since the temperature rise in the target tissue is limited to a safe 42° C. Healthy tissue is not harmed even if nano-particles errantly reside in a healthy cell. In fact, one treatment protocol could be to have nano-particles present in all cells, healthy and cancerous.
The targeting capability of multidimensional radiation technology enables the exact shape of the tumorous region, plus some extended boundary volume, to be treated with radiation. This precision is difficult with other types of treatment technologies.
The Low Temperature Hyperthermia System realizes up to three stepped methods of cancer cell killing: radiation and/or chemotherapy, low temperature hyperthermia, and cytotoxin. This process ensures a very high kill rate and significantly lowers the probability that the cancer reappears after treatment.
The treatment protocol is highly flexible. The order of treatment may be different for a given cancer or person. Some patients may have radiation first and low temperature hyperthermia next; or some patients may be treated with low temperature hyperthermia first, followed by radiation.
Cancer cells that may have realized a low nano-particle uptake concentration can be further treated with a cytotoxin. This is of particular use when the cancer is of a more deadly variety or if it is known that the uptake of a given cancer cell for a given nano-particle type is naturally low.
If nano-particles cannot be used for a given patient, it is possible to use RF- or microwave-based hyperthermia without nano-particles but with very tight temperature feedback controls to realize the target 42° C. in the cancerous tissue and surrounding tissue. In this case, there is no temperature discrimination between cancer and healthy tissue in terms of heating. This approach isn't optimal, since heating fields can cause hot spots in healthy tissue, but it is a fallback if nano-particles can't be used.
Tumors in any location, ranging from on or near the skin to deep in the abdomen or lungs, can be treated easily and safely.
Nano-particles are safely removed by the body's natural filtering systems after radiation and Low Temperature Hyperthermia treatment is complete. Thus, residual nano-particles do not stay in the body.
At 42° C., heat shock protein production is reduced thereby minimizing the level of cancer stem cells/other cells emitted by the resident cancer.
At 42° C., re-oxygenation stresses and kills cancer stem cells because cancer stem cells die in a non-hypoxic environment.

This Low Temperature Hyperthermia System takes advantage of many treatment modalities, each having distinct advantages, wherein the combined treatment protocol is safe and efficacious. The combined approach of multiple killing steps can be further optimized based on the specifics of a given cancer and the individual. This level of flexibility and control has heretofore not been available. The approach taken is one of optimizing the relationship between the exciting energy field and the nano-particle characteristics, where the optimization is in this case one of behavior at a given specified temperature. Certain properties can be designed into the nano-particles to enable a pre-determined temperature rise based on the strength of the energy field: E, H, E and H, EM, acoustical, or optical.

### BRIEF DESCRIPTION OF THE DRAWINGS

FIG. 1 illustrates various protocols for use in Low Temperature Hyperthermia Treatment;

FIG. 2 illustrates, in block diagram form, the typical architecture of the Low Temperature Hyperthermia Treatment System;

FIGS. 3A and 3B illustrate, in flow diagram form, the operation of the Low Temperature Hyperthermia Treatment System to treat invasive agents in a target portion of a living organism;

FIG. 4 illustrates a Hyperthermia Thermal treatment cellular environment;

FIG. 5 illustrates, in table format, the various nano-particle types as paired with field types to realize Low Temperature Hyperthermia;

FIG. 6 illustrates the operation of Low Temperature Hyperthermia using the Magneto-caloric Effect;

FIG. 7 illustrates the operation of Low Temperature Hyperthermia using the Electro-caloric Effect;

FIG. 8 illustrates the operation of Low Temperature Hyperthermia using the Combined Magneto-caloric/Electro-caloric Effect;

FIG. 9 illustrates the operation of Low Temperature Hyperthermia using the Curie Effect;

FIG. 10 illustrates, in table format, the various Low Temperature Hyperthermia effects with corresponding Particle Types;

FIG. 11 illustrates, in graphical form, the Arrhenius Curve which charts Cell Death Probability vs. Cell Temperature;

FIG. 12 illustrates, in flow diagram form, physiological benefits of Low Temperature Hyperthermia;

FIG. 13 illustrates, in flow diagram form, mechanics and modifiers of Hyperthermia Toxicity;

FIG. 14 illustrates, in flow diagram form, lipid shell nano-particle with Cytotoxin Payload;

FIG. 15 illustrates a side view of a table that can be used with the Energy Field and Target Correlation System to irradiate human breast tissue in a human laying prone face down on said table;

FIG. 16 illustrates a side view of an alternative implementation of a table that can be used with the Energy Field and Target Correlation System to irradiate human breast tissue in a human laying prone face down on said table; and

FIG. 17 illustrates additional details of an antenna system that can be used to irradiate human breast tissue in a human laying prone face down on said table.

### DETAILED DESCRIPTION OF THE PREFERRED EMBODIMENT

### Treatment Approaches Used in the Low Temperature Hyperthermia System

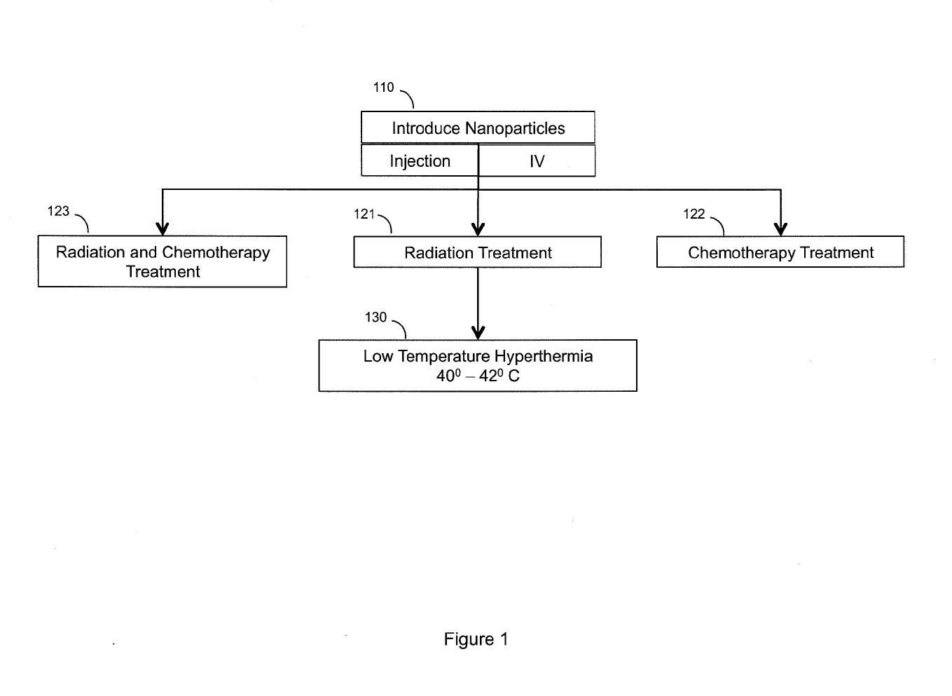

The treatment approaches used in the Low Temperature Hyperthermia System are graphically illustrated in FIG. 1. The Low Temperature Hyperthermia System typically makes use of a pre-treatment process of radiation or chemotherapy (or both) to treat the cancer followed by Low Temperature Hyperthermia (LTH) treatment of the cancer. A first step of the process is where the specially designed nano-particles are delivered into the patient (living organism) by Intravenous (IV) and/or direct Injection at the cancer site (step 110). The application of an energy field causes the nano-particles to rise to a pre-determined temperature that resides in the low temperature hyperthermia region, 42° C. and below. It should be noted that the optimal low temperature hyperthermia region may be different for different people based on many factors. It could also be different for animals, since nothing precludes this treatment paradigm from being used on other living organisms. Separately, different cancer cells may respond optimally to different temperatures and that the Low Temperature Hyperthermia target temperature could vary slightly based on a given cancer, the person being treated, the region being treated and so on. Nothing herein precludes the design of the nano-particles to realize a “not to exceed” temperature of something other than 42° C.

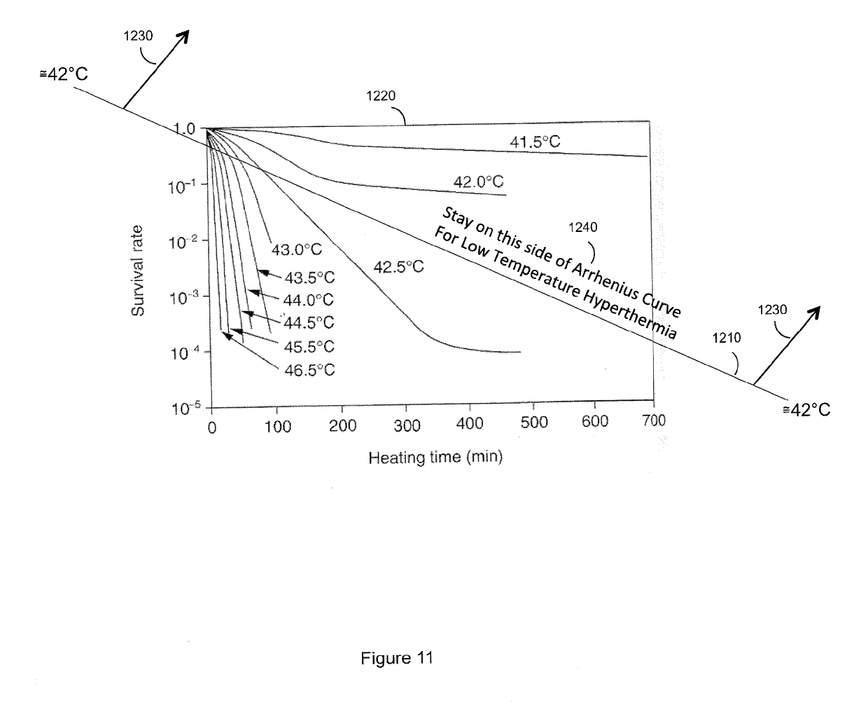

Next, the cancerous region is treated with either radiation at step 121, typically multi-beam radiation that can accurately transcribe the three dimensional extent of the cancerous region, or chemotherapy at step 122 or a sequence of radiation and chemotherapy at step 123. At step 130, Low Temperature Hyperthermia is used to bring the cancerous cells to a predetermined target temperature, typically 42° Celsius. As seen in the Arrhenius Curve, 1220, shown in FIG. 11, the percentage of cell death is very low when the temperature is below 42.25° C. and colder, delineated by line 1210, depicted in region 1220 described by lines 1230. Of note, the Arrhenius curve has been studied both “in vitro” (in the glass) and “in vivo” (in the body) and studies conclude that the cell death probabilities are consistent, whether “in vitro” in a Petri dish or “in vivo” in a breast tissue region, for example. Further stressing the cancer cells already “hit” with radiation in this lower temperature region significantly helps minimize the release of cancer stem cells (which propagate the cancer to other parts of the body). This dramatically improves the odds that the cancer will not reappear.

Note that the steps described in FIG. 1 are one preferred embodiment. The order of treatment could vary, cancer-to-cancer or person-to-person. For example, it may be learned that liver cancer treatment should be Low Temperature Hyperthermia first and radiation second; or it could be learned that radiation and Low Temperature Hyperthermia should be time concurrent, i.e., at the same time. Nothing herein precludes changing the order of when these individual treatment steps are performed.

Note that the time frame between each step can vary, but one of the reasons for pre-administering the nano-particles is to ensure they are on site, residing in cancer cells, so that Low Temperature Hyperthermia treatment can begin immediately after the second to the last step, whether it be radiation or chemotherapy. This ready availability of nano-particles on site, residing in cancer cells, offers enhanced treatment options since nano-particle arrival times can vary.

Examining the basic thermodynamics involved teaches that the nano-particles likely need to be heated to temperature greater than the desired cancer cell temperature. This is due to heat loss thermodynamics, as the nano-particles absorb energy from the illuminating energy field and then transfer that heat to the cancer cell. In the long term, the temperature of the cancer cell approaches that of the nano-particle. The time frame for this to occur involves many variables not discussed herein. Suffice it to say that nothing herein precludes an implementation where the target temperature of the nano-particle is the desired cell temperature, or the target temperature of the nano-particle is some nominal temperature above the desired cancer cell temperature to account for thermodynamic heat losses.

### Architecture of the Low Temperature Hyperthermia System

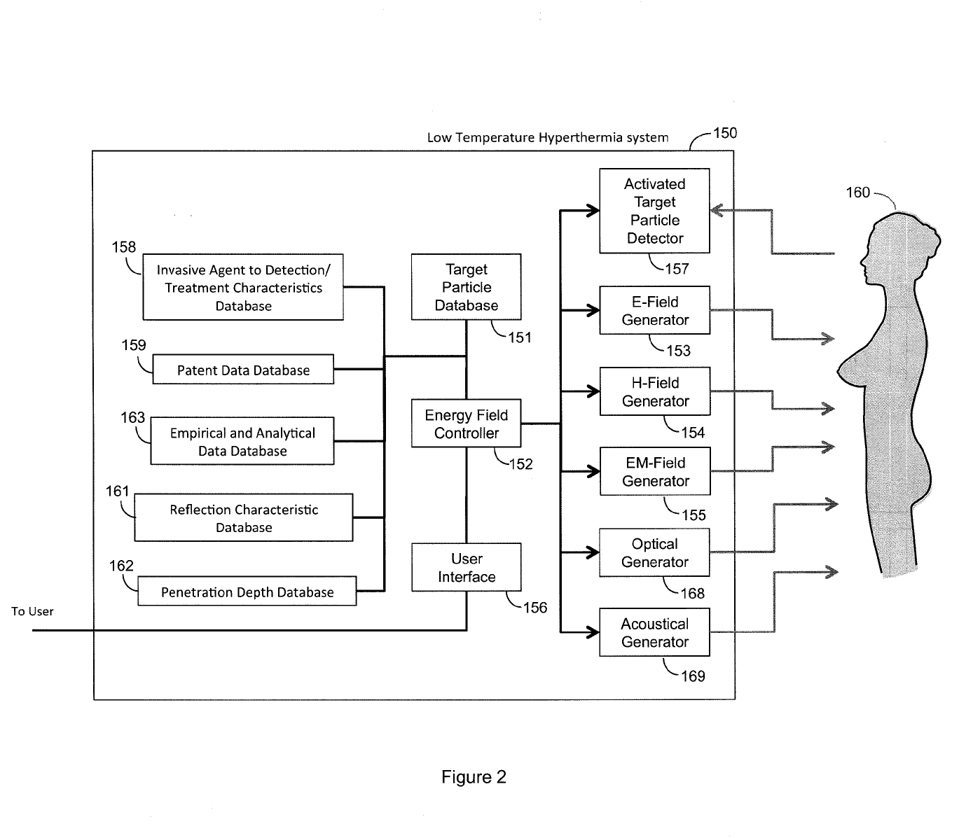

The generation of energy fields to illuminate the nano-particles resident in the cancer cells is achieved by the Low Temperature Hyperthermia System 150 as shown in FIG. 2. In particular, there are a number of databases which maintain information relevant to the illumination process. In particular, a Target Particle Database 151 maintains a listing of characteristics of at least one type of target particle, from the characteristics of target particles including: size, shape, material composition, surface coating, geometry, contents. The Invasive Agent-To-Detection Characteristics Database 158 maintains data which characterizes the relationship between the invasive agent and the characteristics needed to produce the desired target temperature for a selected type of target particle. In addition, Patient Data Database 159 maintains patient-specific data which provides data regarding the age, sex, weight, prior surgeries or other conditions relevant to the treatment process. The Empirical And Analytical Data Database 163 maintains information which has been collected via modeling, testing, theoretical computations, and the like. The Reflection Characteristics Database 161 contains data which represents the percentage of an incident signal which is reflected at the interface between two materials, biological, water, air or the like. Finally, the Penetration Depth Database 162 contains data which represents the attenuation of an incident signal as it passes through a selected material. Databases 161 and 162 are more specifically allocated to E- or EM-Fields, where the E-Field component has certain propagation behaviors at the different tissue layers. In contrast, a magnetic field or H-Field would not have these reflection or penetration values used in its configuration, set-up and illumination calculation by Energy Field Controller 152 (a magnetic field illumination would not use Databases 161 and 162). The number and contents of these databases are selected to illustrate the concepts of the Low Temperature Hyperthermia System 150 and are not intended to limit the application of the Low Temperature Hyperthermia System 150. Some or all of these databases or other data inputs can be used to generate the energy fields to illuminate the cancer cells pursuant to the Low Temperature Hyperthermia paradigm described herein.

There are also one or more Field Generators 153-155, 158, and 159 for generating an energy field. An Electric Field Generator 153 is shown for producing an electric field, a Magnetic Field Generator 154 is shown for producing a magnetic field, an Electromagnetic Field Generator 155 is shown for producing an electromagnetic field, an Optical Generator 158 is shown for producing an optical field, and an Acoustic Generator 159 is shown for producing an acoustical field. Any combination of these Field Generators 153-155, 158, and 159 may be present and can be activated individually or simultaneously, as required. At the outputs of each of these field generators, 153 through 155, there are illumination radiators which may comprise antennas, antenna arrays, magnetic coils, and so on. The purpose of these radiators (not shown in FIG. 2 for clarity) is to provide the output energy field or the energy impulse that excites the tissue and the target nano-particles. The antennas could be linearly polarized such as in horizontal and/or vertical, or they could be elliptically polarized, or they could be circularly polarized such as in Left Hand or Right Hand Circular. The output energy field could be a pulse or series of pulses at RF or microwave frequencies or it could be optical as shown in Optical Generator 158 which is, in practical terms, a laser. Finally, Acoustic Generator 159 could be used if the desired excitation frequency resides more in the acoustical sonic or ultra-sonic region.

An Energy Field Controller 152 is responsive to a user selecting, via the User Interface 15, at least one type of the target particles and identifying a portion of a target living organism which contains these target particles, to automatically select energy field characteristics, from the characteristics of energy fields including: field type, frequency, field strength, duration, field modulation, repetition frequency, beam size and focal point, to energize the selected target particles in a selected manner in the identified portion of the target living organism.

### Positioning Apparatus For Illuminating A Living Organism

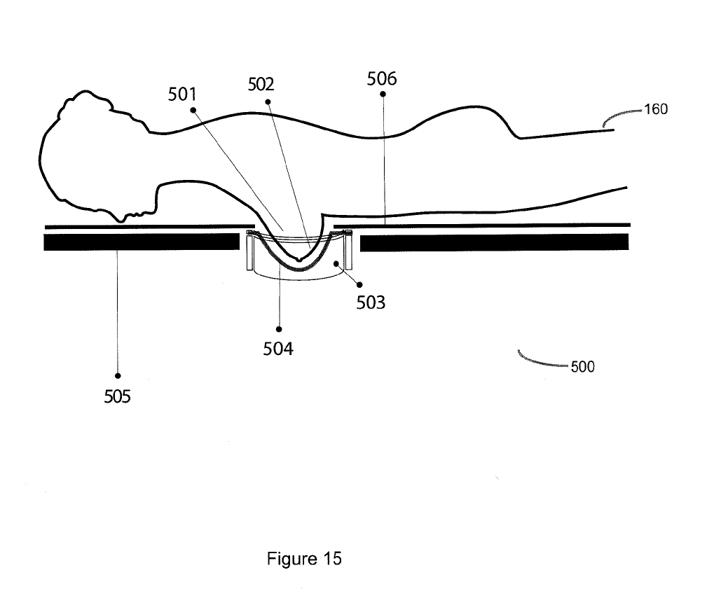

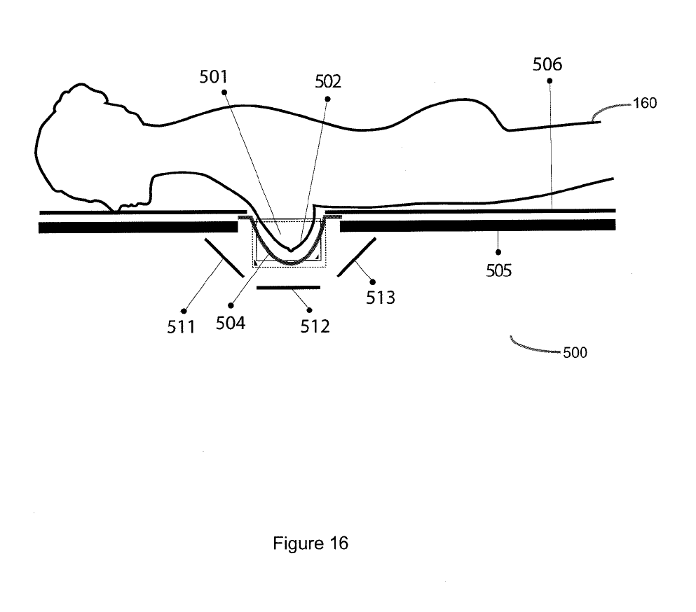

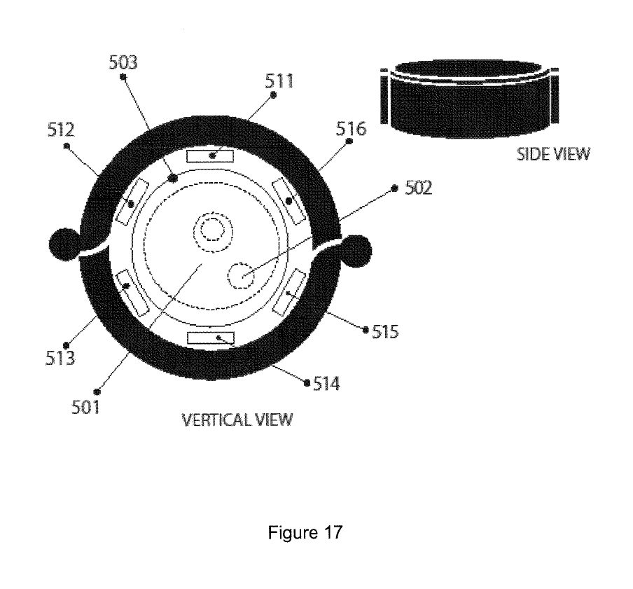

FIG. 15 illustrates a side view of a table 500 that can be used with the Energy Field and Target Correlation System 150 to irradiate human breast tissue; FIG. 16 illustrates a side view of an alternative implementation of a table 500 that can be used with the Energy Field and Target Correlation System 150 to irradiate human breast tissue; and FIG. 17 illustrates additional details of an antenna system that can be used to irradiate human breast tissue using electromagnetic waves.

As shown in these figures, the living organism is a woman 160 who is laying face-down on a table 500, in which an aperture is formed to receive her breast 501 for imaging. As shown, the breast 501 contains a tumor 502 that is the subject of the detection process. In order to minimize the reflections caused by the interface between different materials, a field matching substance 503 (FIG. 15) or an RF matching blanket 504 (FIGS. 15 and 16) is provided to encompass the breast 501 when it is in position between the encircling antennas 511-516 (FIG. 17) and the breast 501. The table 500 can be manufactured from an RF absorbing material 505 to prevent the woman's body from stray RF energy that may emanate from the antennas 511-516. Alternatively, or in addition to, the RF absorbing table, an RF shield 506 can be provided to prevent the woman's body 160 from stray RF energy that may emanate from the antennas 511-516. Typically, there is a plurality of radiating elements 511-516 used to implement the antenna, as shown in FIG. 17, and are positioned to encircle the breast 501.

A matching “blanket” or material is used to match the electric field or magnetic field or electromagnetic field to the tissue. The skin is the first barrier and has a typical dielectric constant, ranging from 1000 at 1 MHz to 80 at 1 GHz. The respective conductivity at 1 MHz is 0.01 S/m and at 1 GHz is 0.8 S/m (Siemens/meter). Moistening the skin with an aqueous solution of NaCl changes the conductivities below 100 MHz but sees little to no change for the permittivity of wetted skin. If the energy is delivered by free space, as from an antenna, the electric field (EM-Field) needs to be matched to the skin layer to minimize the reflection off of the skin boundary condition. A simple matching “circuit” or material is 90 electrical degrees long at the center of the selected frequency band. Multiple matching circuits or layers can be used to enhance the bandwidth of the match over a broader frequency range. In general, the quarter wave transformer (90 electrical degrees long) matches from one medium to a second medium. Classically, the impedance of the matching medium is the square root of the product of the end point impedances. This impedance matching is less critical for a pure magnetic or H-Field.

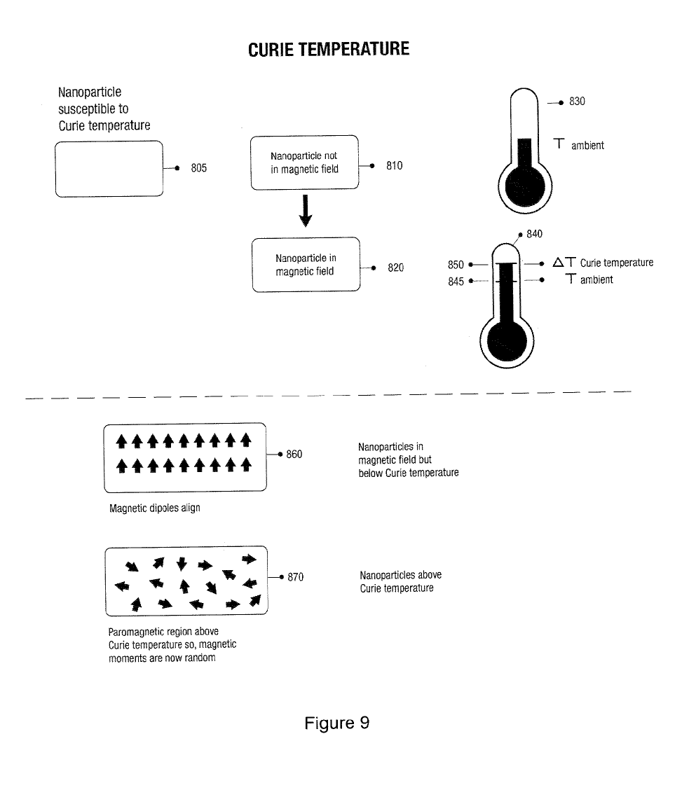

In FIG. 16, the antennas or radiators contained within devices 511, 512, and 513 are connected physically to the outputs of the Energy Field and Target Correlation System as shown in FIG. 1A at the output arrow lines of generators 103, 104, 105, 108, and 109. These antennas take the energy from the field generators and illuminate the breast tissue with a pulse of energy or continuous energy in the form of an E-Field, an H-Field, or an EM-Field to include an Optical Generator 108 which is a laser for skin cancer (example) or Acoustical 109 such as for an ultrasonic transducer. In addition, in FIG. 9 at devices 511, 512, and 513, these devices also contain ultrasonic or acoustical receive detectors to pick up the acoustical signature of the tissue and particles under pulsed excitation. Separately, devices 511, 512, and 513 also offer a means to detect thermal or temperature differences as described herein. These inputs or receive signals are sent to device 107 in 150 (the Activated Target Particle Detector). Additional detected signals include material properties responses of healthy tissue, cancerous tissue, and nano-particles.

In FIG. 17, devices 511, 512, 513, 514, 515, and 516 embody similar functionality. They serve as radiating antennas or elements for the generators in Device 150 (103, 104, 105, 108, and 109) and they serve as receiving or pick-up sensors or antennas for Activated Target Particle Detector 107 to detect or sense:
the acoustical response (from photo or thermal acoustic excitation);
the thermal response (from continuous or pulsed generator excitation);
the materials properties response (from continuous or pulsed generator excitation);
and so on.

In FIG. 17, element 501 is the human breast while element 502 is a cancerous lesion being imaged. Lesion 502 has nano-particles resident inside the cancer cells offering a contrast agent for the imaging methods described herein: photo/thermal acoustic, materials properties and quasi-steady state thermal rise.

### Feedback

There are a number of logical feedback loops, where the feedback enables the Low Temperature Hyperthermia System 150 to have an optimum response. For example, feedback from an image is used to enable optimal treatment. Feedback from a fuzzy image could be enhanced by feedback telling the Low Temperature Hyperthermia System 150 to re-image the spatial boundaries of the cancer's extent. Feedback during treatment ensures that nano-particles are heated to the desired temperature, 42° C. for certain applications, and significantly higher to kill the cancer cells. This feedback largely takes place between the Activated Target Particle Detector 107 and the Energy Field Controller 102 of the Low Temperature Hyperthermia System 150.

Thus, the user inputs data relating to the class of target particles and the portion of the living organism that is being treated, which causes the Energy Field Controller 152 to automatically determine the appropriate set of energy field characteristics, which are required for application to the designated portion of the target living organism to activate the target particles to respond in a detectable manner to enable the identification, via an Activated Target Particle Detector 157, of a presence, locus and response of the target particles in the living organism (as disclosed in further detail below). The Energy Field Controller 152 uses the automatically determined set of energy field characteristics to activate the corresponding Energy Field Generator(s) 153-155, 158, and 159 to output the corresponding energy fields as defined by the set of energy field characteristics. It should be noted that an automated system improves accuracy and prevents human imaging errors; but nothing herein prevents the Low Temperature Hyperthermia System 150 from being operated in a manual form, should a special case arise wherein a manually entered algorithm could potentially enable higher imaging contrast or resolution; or a more efficacious treatment protocol.

### Operation of the Low Temperature Hyperthermia System

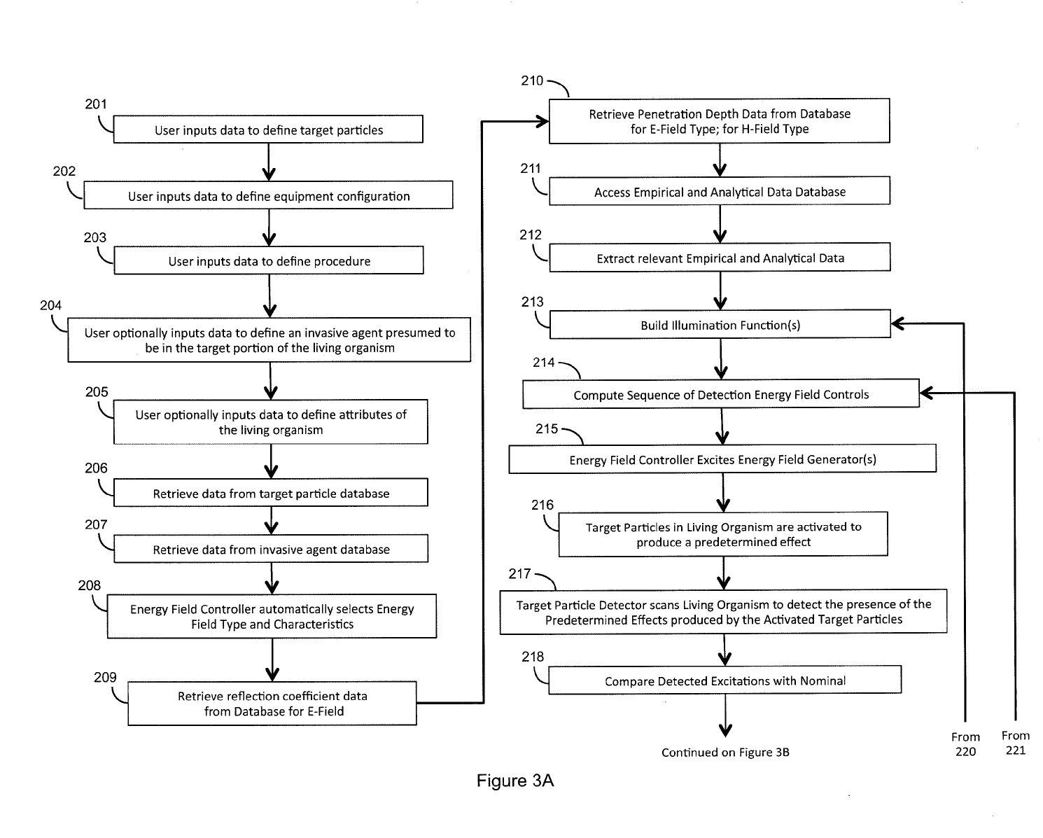

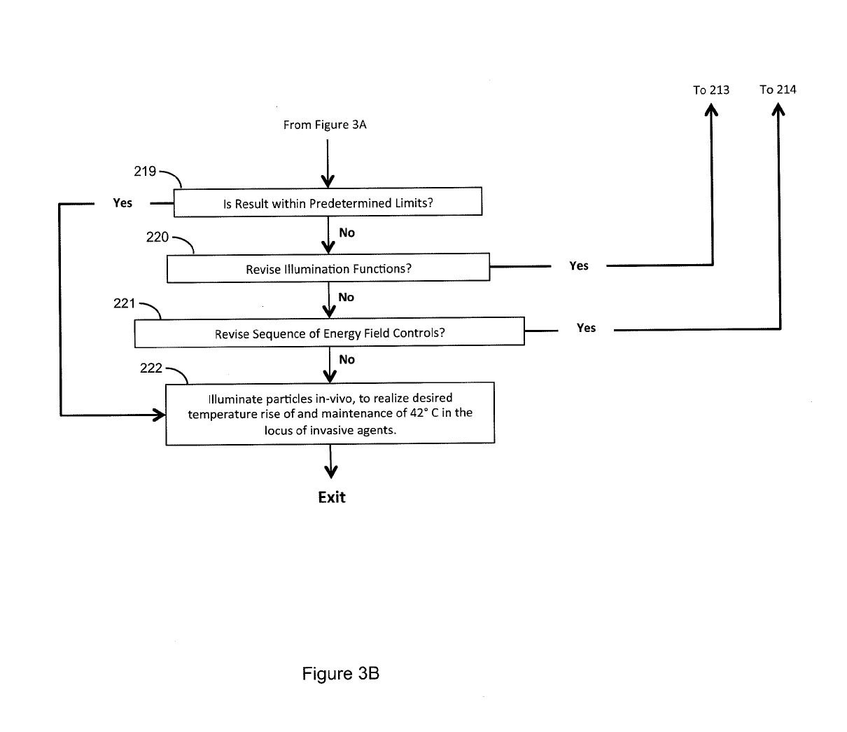

FIGS. 3A and 3B illustrate in flow diagram form the operation of the Energy Field and Target Correlation System 150 to generate the energy fields used to illuminate invasive agents in a target portion of a living organism as well as treat the detected invasive agents via the use of Low Temperature Hyperthermia. The Low Temperature Hyperthermia System 150 receives a set of user provided input data to define the protocol, equipment configuration, living organism as well as the target particles that have been deployed in the living organism. This data is then used by the Low Temperature Hyperthermia System 150 to automatically build a set of illumination functions and compute the sequence of energy field controls that are required for the invasive agent detection and treatment protocols. In addition, the Low Temperature Hyperthermia System 150 makes use of dynamic feedback to adjust the energy fields during the execution of a selected protocol.

At step 201, the user inputs data via User Interface 156 to the Low Temperature Hyperthermia System 150 to define target particles deployed in the living organism 160, such as in the breast of the woman 160. At step 202, the user optionally inputs data via User Interface 157 to the Low Temperature Hyperthermia System 150 to define the configuration of the equipment. If the equipment configuration is invariant, this step can be skipped. The user can also input data via User Interface 156 to the Low Temperature Hyperthermia System 150 to define the procedure being executed, such as a detection procedure or a treatment procedure or a combined detection and treatment procedure. The user can then input data into the Low Temperature Hyperthermia System 150 at step 204 via User Interface 156 to define an invasive agent (such as breast cancer) presumed to be in the target portion of the living organism 160. At step 205, the user optionally inputs data via User Interface 156 to the Low Temperature Hyperthermia System 150 that identifies a selected living organism 160 and the attributes of this living organism 160. This pairing of input information defines the particular application that must be addressed by the Energy Field Controller 152 in automatically generating an illumination protocol that is effective for this application, yet not excessive and potentially damaging to the living organism 160.

In response to these data inputs, at step 206, the Energy Field Controller 152 retrieves data from the Target Particle Database 151 and, at step 207 the Energy Field Controller 152 retrieves data from the Invasive Agent Database 158. This retrieved data, in conjunction with the user input data is used by the Energy Field Controller 152 at step 208 to automatically select energy field characteristics; this also could be set manually, depending on specific circumstances. The energy field characteristics include: field type, frequency, field strength, field modulation, repetition frequency, beam size and focal point, and the like. These energy field characteristics are needed to produce a precisely crafted energy field with is mapped to the target particle characteristics and the target portion of the living organism 160.

At step 209, the Energy Field Controller 152 optionally retrieves reflection coefficient data from the Reflection Characteristic Database 161 and also retrieves penetration depth data at step 210 from the Penetration Depth Database 162 (this is for an E-Field component; the H-Field excitation is less susceptible to these issues as previously discussed herein). This data enables the Energy Field Controller 152 to account for the particular tissues that the generated energy fields will traverse to reach the deployed target particles. This information is used to adjust the selected energy field characteristics as computed at step 208.

At step 211, the Energy Field Controller 152 optionally accesses the Empirical And Analytical Data Database 163 that maintains information which has been collected via modeling, testing, theoretical computations, and the like. This data represents the experiential knowledge that can be used by the Low Temperature Hyperthermia System 150 to automatically set the illumination functions and energy field generator controls. Thus, at step 212, the Energy Field Controller 152 extracts whatever data is relevant to the proposed protocol from the Empirical And Analytical Data Database 163. This step completes the data input, collection, and extraction functions.

At step 213, the Energy Field Controller 152 proceeds to automatically build a set of illumination functions which are used to destroy the invasive agents in the living organism. These illumination functions are then used by the Energy Field Controller 152 to compute a sequence of energy field controls, which are the control signals used to activate selected Energy Field Generators 153-155 to produce the energy fields necessary to activate the target particles to produce a desired and detectable effect via the application of the energy field controls at step 215.

The Energy Field Generator(s) produce one or more energy fields corresponding to the selected energy field characteristics to illuminate the target portion of the living organism 160 and at step 216, the target particles in the living organism are activated to produce a predefined effect which can be detected at step 217 by the Activated Target Particle Detector 157 and which enable differentiation between the activated target particles in their associated invasive agents and the surrounding normal cells in the living organism. For some specific nano-particle designs, where the nano-particle does not heat past a certain temperature, say 42° C., then this “detection” step 217 may not be required. Then, at step 218, the Activated Target Particle Detector 157 compares the detected excitations with what is expected and at step 219 determines whether the detected effects are within predetermined limits. If so, the Activated Target Particle Detector 157 advances to step 222 where the process may reside for a given period of time to keep the cancer and cancer stem cells at the nominal 42° C. for the determined time frame to ensure the desired effect is realized. This “bake” time at 42° C. could vary for different cancers or cancer locations. The programming of Energy Field Controller 152 would contain these “bake” time frames in the various databases.

If the Activated Target Particle Detector 157 determines at step 219 that the detected effects are not within predetermined limits, processing advances to step 220 where a determination is made whether the illumination functions need to be adjusted by routing back to step 213. If not, processing advances to step 221 where a determination is made whether the detection energy field controls need to be adjusted by routing back to step 214. If not, processing advances to step 222 as described above until the treatment process has completed, and the process exits.

### Thermodynamic Profile of a Cancer Cell Being Illuminated

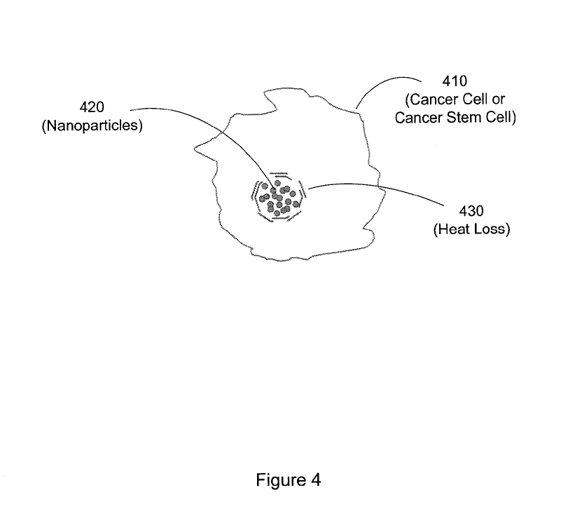

FIG. 4 is an example of a representative thermodynamic profile of a cancer cell being illuminated by an energy field. Nothing herein precludes some other thermodynamic profile. The example used here is merely representative, as are the other examples used in the remainder of the figures in this specification. For example, nano-particle clumping in the cancer cell may require the use of one nano-particle target temperature while a more uniform nano-particle distribution in the cell may require a different nano-particle target temperature; all to realize an overall nominal cell target temperature of 42° C. or colder. The nano-particle behavior in the cancer cell, clumped or not clumped, can be controlled to some degree by using surfactants and nano-particle coatings to reduce the tendency of the nano-particles to clump. In addition, nano-particle size greatly affects the tendency to clump. Nano-particles that are small, say less than 10 nanometers, tend to clump to reduce the overall surface energy state of the nano-particle mass. This is because small nano-particles have a greater number of their atoms near the nano-particle's physical surface. For example, a nano-particle that is around 2 nm, shape dependent, has all of its atoms on the nano-particle surface. A 10 nm nano-particle, again shape dependent, has around 50% of its atoms on the surface.

Thus, nano-particle size and nano-particle coating can greatly determine the propensity to clump or not to clump in the cancer cell. Other nano-particle characteristics such as three dimensional shapes can impact clumping. There may be times where clumping is desired and the corresponding nano-particle target temperature is designed accordingly; alternatively, for a given cancer cell, it may be more desirable to have a more uniform nano-particle distribution in the cancer cell with a different nano-particle target temperature. Thus, nano-particle design methods are used to reduce the propensity or tendency to clump; or, they are used to enhance the propensity or tendency to clump. Thus, either state, clumped or not clumped, could be optimal, with the nano-particle target temperature designed accordingly for each “clumping state”.

A cancer cell 410 (or cancer stem cell) has a locus of nano-particles resident 420. When the nano-particles 420 are heated by the external energy field, a heat transfer loss occurs at 430 between the nano-particles and the cancer cell. In order to realize an optimal temperature distribution across the cancer cell's extent, where such temperature profile is somewhat dependent on whether the nano-particles have clumped in the cancer cell, the target temperature of the nano-particle could be the same as the target temperature of the cancer cell or it could be different to account for the thermal loss between the nano-particles and the cancer cell. In this example, the nano-particles are heated to a temperature higher than that of the cancer cell due to a thermal loss at the particle/cell interface, where the heat loss is shown as 430. To determine the nano-particle temperature, the desired cancer cell temperature and the loss parameters are determined. In this example, the desired cancer cell temperature is 42° C. and that is equivalent to the nano-particle temperature minus the temperature loss. Thus, the nano-particle temperature in this simple example is determined by:

Temp_{particle}≅42° C.+Heat Loss

Other thermodynamic equations would come into use for more complex heat loss or heat transfer scenarios.

### Methods of Controlling Nano-Particle Temperature

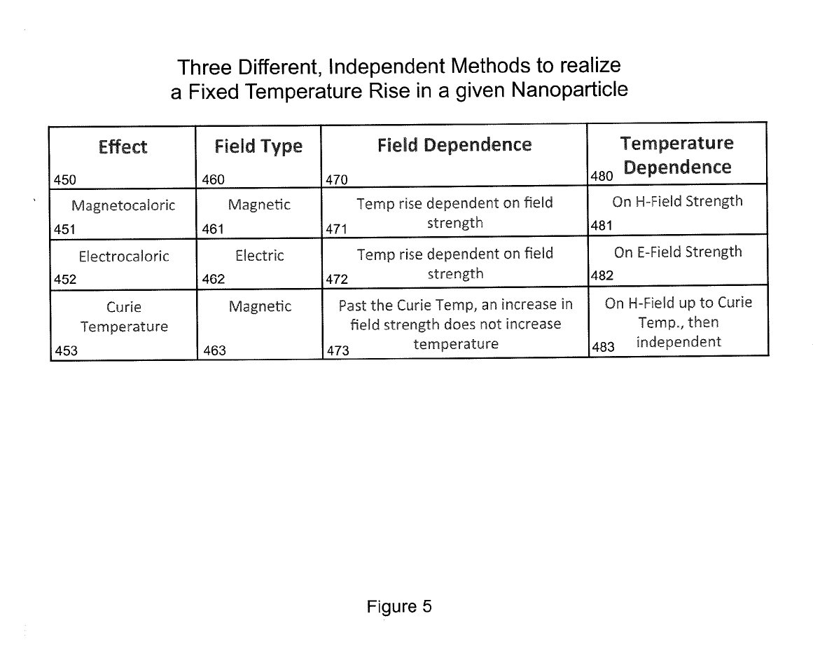

There are at least three methods for accurately controlling the nano-particle temperature: the Curie temperature, the magneto-caloric effect and the electro-caloric effect. As shown in FIG. 5, there are minimally four attributes of interest: the Effect (450), the Field Type (460), the Field Dependence (470) and the Temperature Dependence (480). For the Effects (450), there are minimally three approaches to realize a controlled temperature rise in a nano-particle: the Magneto-caloric Effect (451), the Electro-caloric Effect (452) and the Curie Temperature (453). Now, looking horizontally, the attributes of each Effect can be studied. For the magneto-caloric effect, the field type is Magnetic (461) and the field dependence is Field Strength (471) with temperature dependence on H-Field Strength (481). Similarly, for the electro-caloric effect, the field type is Electric (462) with the field dependence being Field Strength (472) and the temperature dependence on E-Field Strength (482). Last of the three, Curie temperature, has a field type of Magnetic (463) with a field dependence of a Field Strength Cut-off (473) and a temperature dependence of a given H-Field strength and nothing higher.

Alternatively, it is possible to use a heating method where “regular” nano-particles that heat up in a field, whether the field is electric or magnetic of a combination of the two, are used to heat up cancer cells. This approach does not have the precision of using specially designed nano-particles. Some feedback mechanism must be employed to accurately manage the applied energy field to not exceed the desired cancer cell temperature such as that at step 217 in FIG. 3A, “detects the predetermined effect.” This is a very complex process, albeit not impossible, that requires some way of accurately measuring the temperature of the cancer cell. The energy field excitation must be anticipated to not overshoot the heating of the cancer cell to a non-Low Temperature Hyperthermia range. For cancers other than skin cancer, this could be very complex and potentially not very accurate.

### Magneto-Caloric Effect in the Low Temperature Hyperthermia System

The Magneto-caloric Effect was originally envisioned for magnetic cooling or refrigeration. Since the magneto-caloric effect's cooling stage happens after the magnetic field is removed, it can be used to bring substances very close to absolute zero (after the initial ambient heat rise is removed by other environmental cooling means). This is called adiabatic demagnetization.

The Magneto-caloric Effect heating during the adiabatic magnetization phase is due to the application of a Direct Current (DC) magnetic field. This is in contrast to the heating of ferromagnetic particles in an Alternating Current (AC) magnetic field. This is an important distinction between the multiple methods described herein which are used to heat nano-particles to a given temperature, Magneto-caloric is a DC magnetic field while particles in the ferromagnetic state are best heated using an AC magnetic field.

What is of particular interest to the cancer treatment envisioned herein is the precise rate of temperature rise when magneto-caloric materials are subjected to a magnetic field of given strength, measured in Amps per Meter. While “regular” nano-materials such as iron ferrite Fe₃O₄ heat in an Alternating Current magnetic field, where the frequency of the magnetic field varies from hundreds of kilohertz to megahertz, the rate of temperature rise is less precisely correlated to magnetic field strength. For iron ferrite in a high frequency magnetic field, the nano-particle heats up and the heating is correlated to magnetic field strength, although the heating is not specifically correlated to a set number of degrees of temperature rise for a given increase in magnetic field strength (such as the case for Magneto-caloric nano-particle in a DC field of a given field strength). For iron ferrite, the linear, squared, or cubed relationship to the magnetic field is prevalent as it relates respectively to being in the Brownian, Ned, or Rayleigh magnetic regions (Rayleigh can be both squared and cubed, variable dependent). Thus, an iron ferrite particle could be used but it does not have the precise heating characteristics of a magneto-caloric nano-particle.

Certain materials exhibit the Magneto-caloric Effect. One such chemical element is gadolinium, which is also used in an alloy form as a contrast agent in Magnetic Resonance Imaging (MRI). Thus, this material is safe for use in humans and simply needs to be processed in nano meter dimensions. The gadolinium alloy Gd₅(Si₂Ge₂) has a much stronger Magneto-caloric Effect. Praseodymium alloy with nickel PrNi₅ has a very strong Magneto-caloric response, so strong that it has enabled temperatures to within one thousandth of a degree of absolute zero. This particular “cooling” application is somewhat different from the approach described herein.

The Low Temperature Hyperthermia System 150 uses the Adiabatic Magnetization stage of magnetic cooling, wherein the nano-particles exhibiting a Magneto-caloric Effect residing in a cancer cell then are exposed to a magnetic field with specific field strength. This field strength is determined a priori for the given particle's material composition based on a specified desired temperature rise. The magnetic field causes the magnetic dipoles of the atoms to align, which means the particle's magnetic entropy must decline (go down). Since no energy is lost yet, thermodynamics teaches us that the nano-particles' temperature must go up. It is this very tightly controlled temperature rise, based on a given magnetic field strength, which is of great interest in realizing Low Temperature Hyperthermia.

Clearly, for the cancer cell treatment application of low temperature hyperthermia, what is desired is a nano-particle fabricated from a material that offers around 5° C. to 10° C. of temperature rise in a reasonable magnetic field. Since the normal temperature of the human body is around 37° C., to reach a nominal cellular target temperature of 42° C. plus some heat loss, the nano-particle must be capable of a 5° C. to 10° C. temperature rise in a specified magnetic field. For example, 37° C. ambient body temperature plus 10° C. of nano-particle temperature rise yields a nano-particle temperature of 47° C. Then subtract 5° C. of thermal loss in this example, to yield a cancer cell temperature of 42° C. Other levels of thermal loss are possible and are used in this document as other examples.

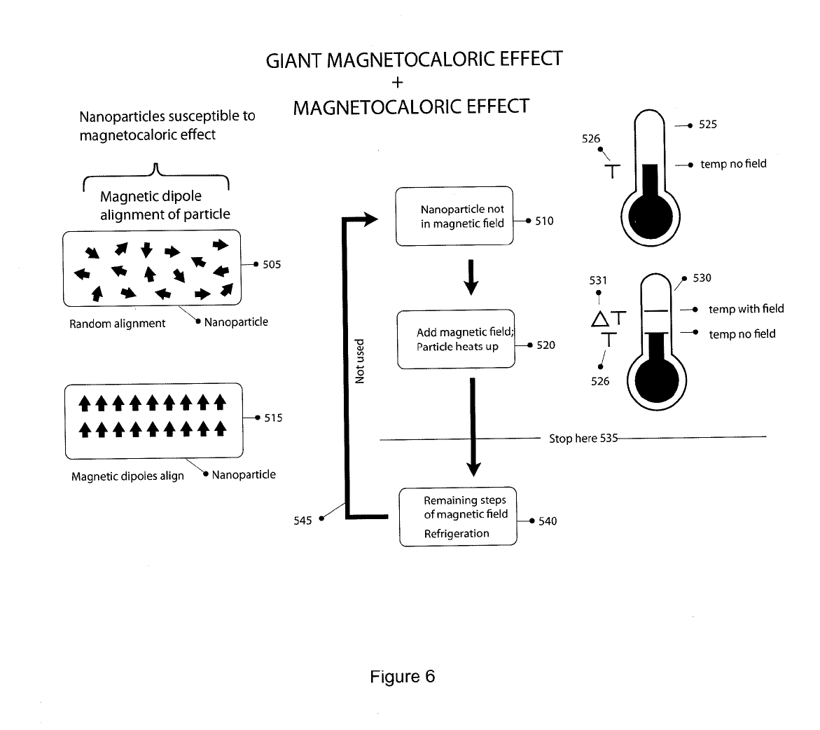

For the Magneto-caloric Effect, as shown in FIG. 6, nano-particles are designed to exhibit this effect at the desired field strength and per degree temperature rise correlation. As illustrated in element 505, the magnetic dipoles of the nano-particle exhibit random alignment when not in the presence of a magnetic field. As illustrated in element 515, when exposed to a magnetic field, the magnetic dipoles of the nano-particle align and nano-particle heating occurs at a specified rate per the applied magnetic field strength; the rate of heating is measured in degrees per incremental field of some value. The process described herein uses a portion of the magnetic refrigeration cycle and discards the unneeded steps of the cycle. Thus, at step 510, the nano-particles are located in the cancer cell, but are not in a magnetic field, the magnetic field is off. Thus, the nano-particle temperature is at ambient, which is the temperature of the cancer cell. This is illustrated in elements 525 and 526. When the magnetic field is applied to the cancerous region, the nano-particles in the cancer cells have their magnetic dipoles align at step 520. The temperature rise is specified by the magneto-caloric effect's properties and the rise is shown as level 531 is illustrated in element 530 (ambient temp was level 526). The Low Temperature Hyperthermia System 150 achieves a tightly controlled thermal rise based on the magnetic field's exciting strength at the region or locus of the cancer cells where the nano-particles reside, under the precise control of the Low Temperature Hyperthermia System 150. Since the remaining steps of the magnetic refrigeration process are not needed, the process terminates at step 535, and steps 540 and 545 are not executed.

For room temperature adiabatic magnetization heating, a number of materials exhibit properties of interest; most are alloys of gadolinium. This is advantageous since gadolinium alloys are being used as contrast agents for MRIs, meaning the material has been approved for use in humans. Gadolinium is strongly paramagnetic at room temperature and exhibits ferromagnetic properties below room temperature. It's Curie temperature, as a pure element, is 17° C.-above 17° C., gadolinium is paramagnetic meaning it only has magnetic properties when it is placed in a magnetic field (the magnetic spins or dipoles are random until a magnetic field is applied). Alloys of gadolinium may have different Curie points. Gadolinium exhibits a magneto-caloric effect where its temperature rises when placed in a DC magnetic field and the temperature decreases when it is removed from the DC magnetic field.

### Electro-Caloric Effect in the Low Temperature Hyperthermia System

Similarly, for the Electro-caloric effect, when a specially designed nano-particle, which exhibits an electro-caloric effect, is placed in a DC electric field, the temperature rise of the nano-particle is dependent on the field strength of the electric field Like the magnetic cooling cycle, the Low Temperature Hyperthermia System 150 uses the first steps of the process and does not use the remaining cooling steps Like the magneto-caloric effect with magnetic fields, the electro-caloric effect realizes a specified temperature increase when exposed to an electric field. As an example material, PZT, a mixture of oxygen, zirconium, lead, and titanium with a 12° C. temperature response in a field voltage as low as 25 volts—the ambient temperature in this example was 220° C. At room temperature, ferroelectric polymers have shown 12° C. of temperature change when exposed to a DC electric field. Sometimes this effect is called the Giant Electro-caloric Effect.

Of note, since the electric field is DC, no tissue is heated by this DC field. In contrast, an AC electric field heats a non-electro-caloric particle as well as tissue, if the excitation frequency is greater than a few hundred MHz, where the dipolar nature of the water in the tissue causes the polarized water molecule to rotate and, therefore, cause frictional heat. Thus, the Electro-caloric particle in a DC electric field has the advantage of zero unintentional tissue heating.

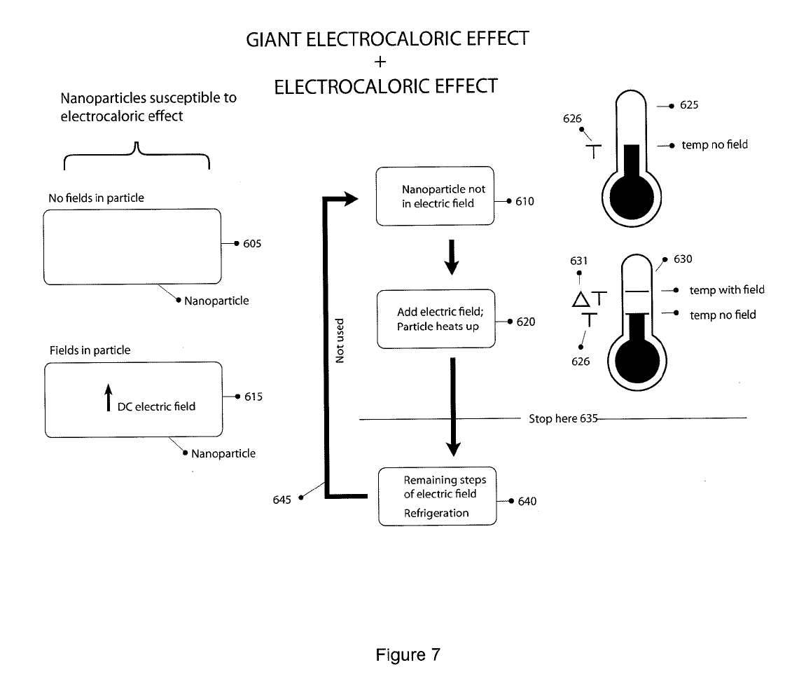

FIG. 7 shows the electro-caloric effect. As illustrated in element 605, a nano-particle is shown not in an electric field while the nano-particle is illustrated in element 615 as in the electric field. At step 610, the nano-particle is not in the DC electric field and has an ambient temperature of level 626 is illustrated in element 625. When the DC electric field is applied to the nano-particle at step 620, the temperature rises to ΔT at level 631 which is greater than ambient temperature of T at level 626 (is illustrated in element 630). The remaining steps of the electro-caloric cooling process, steps 640 and 645, are not used and the process stops at step 635. Of course, like the magnetic cooling process, the electric cooling process has additional steps which offer cooling to cancer cells—for now, only heating is desired.

### Combined Magneto- and Electro-Caloric Effect in the Low Temperature Hyperthermia System

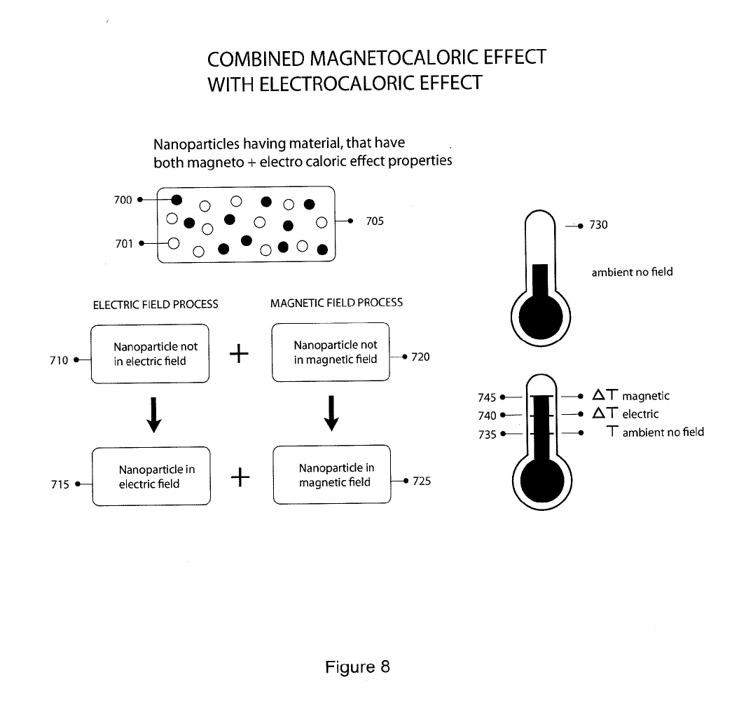

FIG. 8 illustrates the use of a nano-particle 705 that is susceptible to both Magneto-caloric 700 and Electro-caloric 701 Effects. When the nano-particle is located in the body and is not in an electric field as illustrated in element 710 and not in a magnetic field as illustrated in element 720, the ambient temperature of level 735 (T) is realized. When the nano-particle is illuminated by an electric field as illustrated in element 715 and a magnetic field as illustrated in element 725, the corresponding temperature rise in the nano-particle has two components, one from the electric field nano-particle response as indicated by level 740 ΔT_{Electric} and the second from the magnetic field response as indicated by level 745 ΔT_{Magnetic}. These two responses create or enable a “doubling” of the temperature rise over ambient. Both of these fields, magnetic and electric, are DC in nature.

### Curie Temperature

The Curie temperature of a material is the physical temperature where the material transitions from a ferromagnetic state to a paramagnetic state. Below the Curie temperature the material is ferromagnetic; above the Curie temperature, the material is paramagnetic. This means that the magnetic dipoles or spins of the atoms of the material go from an aligned, ordered state (ferromagnetic) to a purely random state (paramagnetic) (in the absence of an applied magnetic field). This effect is reversible in certain materials as the material moves back and forth across, or above and below, the Curie temperature.

Above the Curie temperature, the thermal energy overcomes the ion magnetic moments resulting in disordered or random magnetic dipoles (the spins) and the material is no longer ferromagnetic, it is now paramagnetic. Paramagnetic materials, in absence of a magnetic field, do not exhibit any magnetic effect. Paramagnetic materials, even in the presence of a magnetic field, only have a relatively small induced magnetization because of the difference between the number of spins aligned with the applied field and the number of spins aligned in the opposing direction; only a small percentage of the total number of spins are oriented by the field flux lines.

How does a nano material behave when in a magnetic field when the temperature is above the Curie point and it is now paramagnetic? This depends on whether the magnetic field is AC or DC. Below the Curie temperature, a ferromagnetic material in an Alternating Current (AC) magnetic field results in nano-particle heating. This is due to the “forced” alignment and re-alignment of the magnetic dipole with the phase of the magnetic field; as the phase changes with time (AC), the dipole attempts to re-align. This creates heating in the ferromagnetic nano-particle. If this field were DC, or a static magnetic field, no steady state heating would occur.

Above the Curie temperature, the material is now paramagnetic. This means the magnetic dipoles are random in the nano-particle. When placed in a DC field, no steady state heating occurs. When placed in an AC or Alternating Magnetic field, there is only a small fraction of the magnetic dipoles or spins that are affected, meaning the “induced” magnetization is low. This is proportional (linear) to the applied field strength. Since the magnetic dipole re-ordering is not anywhere near the magnitude of the magnetic dipole re-ordering in a ferromagnetic particle in an AC magnetic field, the heating of a paramagnetic material, past its Curie temperature, is considerably less.

Some paramagnetic materials are also magneto-caloric; but only a few. Magneto-caloric materials are paramagnetic with special behavior associated with being Magneto-caloric. This should not be confused with materials that are hotter than their Curie temperature and have now become paramagnetic. This particular paramagnetic state is not Magneto-caloric.

Magnetic materials of a certain design exhibit a Curie temperature effect, wherein after a certain magnetic field strength is realized, the material (or nano-particle in this case), the material no longer continues to heat. Paramagnetic materials, even in the presence of a magnetic field, only have a relatively small induced magnetization because of the difference between the number of spins aligned with the applied field and the number of spines aligned in the opposing direction; only a small percentage of the total number of spins. The paramagnetic spins still align along the field lines, but there are not that many that have to be flipped when the field direction is reversed.

The temperature at which this occurs is material dependent and, thus, can be designed to occur at specific temperatures, offering a means to precisely control cancer cell heating. As illustrated in element 805, a nano-particle is shown which is susceptible to heating as a result of being exposed to a magnetic field. As illustrated in element 810, the nano-particle is not in the magnetic field (i.e. the field is turned off) and the nano-particle temperature is stable with its ambient surroundings as illustrated in element 830. For the nano-particle that has been introduced into a cancer cell, this temperature is approximately the ambient body temperature of 37° C. (as illustrated in element 830). When a magnetic field is applied as illustrated in element 820, the nano-particle heats until the Curie temperature is reached wherein the heating essentially stops. This is illustrated as level 850 in element 840. The ambient temperature of level 845 is elevated to a new temperature of level 850, which shows the temperature rise due to the Curie temperature of the nano-particle material.

### Thermal Response to Low Temperature Hyperthermia System

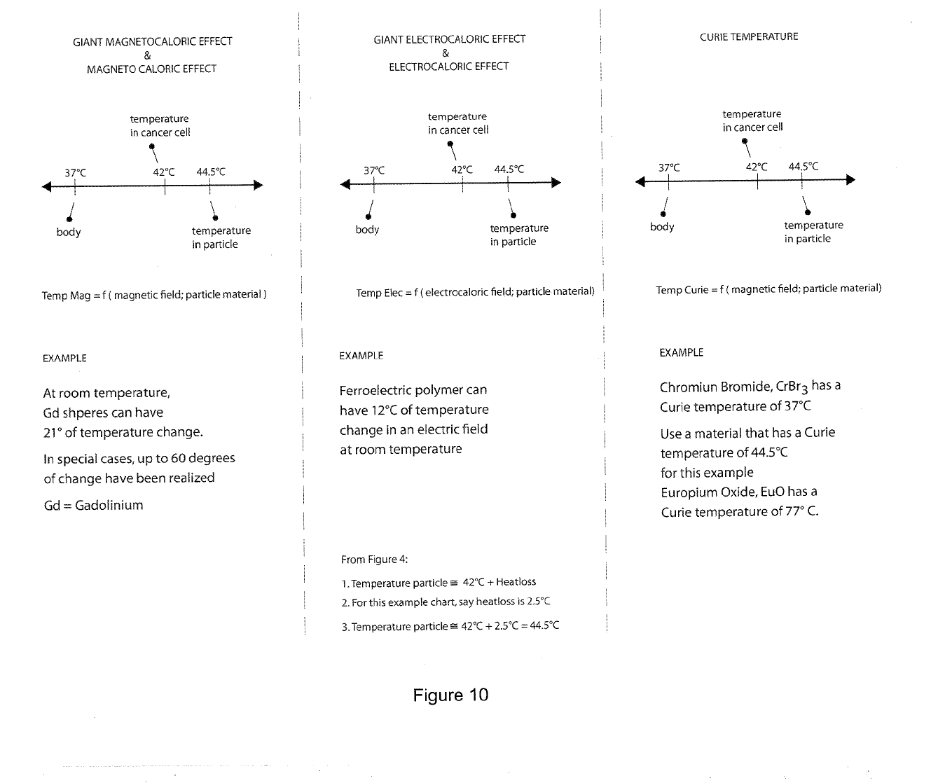

FIG. 10 graphically shows the temperature rise for the three effects just described: magneto-caloric, electro-caloric and Curie. In the far left column, the magneto-caloric effect is shown with the body temperature at 37° C., the particle at 44.5° C. and having thermodynamic losses of 2.5° C. to produce the resultant temperature in the cancer cell of 42° C. This value of 42° C. resides in the low temperature hyperthermia range as shown in FIG. 11 by lines 1210 and 1230 for the cancer cells, which is highly desirable for reasons stated herein, to include the minimization of the release of cancer stem cells. Gadolinium has been shown to have strong magneto-caloric effect with 21° C. of temperature change starting at room temperature or around 21° C. (70° F.). Gadolinium has been shown to support up to 60° C. of temperature change. In the magneto-caloric example, the magnetic nano-material rises 1.5° C. per 3 kA/m of magnetic field. By using the temperatures just discussed, we need 7.5° C. of temperature rise over ambient. This means that the magnetic field needed is 15 kA/m, as shown in the following calculation:

(7.5° C.*3 kA/m)/1.5° C.=15 kA/m

In FIG. 10, in the center column, the Electro-caloric effect is shown with the same temperature ranges as the magnetic example, where the temperature here is a function of the electric field and the nano-particle material. The target cancer cell temperature is 42° C. and a nano-particle exhibiting 2° C. temperature rise per 0.75 kV/m electric field strength requires a total DC electric field strength of 2.81 kV/m in order to realize the desired particle temperature rise of 7.5° C. as shown in the following calculation:

(7.5° C.*0.75 V/m)/2.0° C.=2.81 kV/m

This raises the temperature of the nano-particle from ambient of 37° C. to 44.5° C. less 2.5° C. of loss to arrive at the target temperature of 42° C. for the cancer cells. An example electro-caloric material is a ferroelectric polymer which has up to 12° C. of temperature change at room temperature.

The far right column in FIG. 10 illustrates the Curie Temperature process. At a temperature of 44.5° C., it is desired to have the nano-particle heating largely stop at the Curie point of 44.5° C. The nano-material is selected to have this temperature characteristic. Thus, for example, the magnetic field strength (DC) may be raised to 25 kA/m even though the Curie point is reached with a magnetic field of 20 kA/m. This small overage of field strength insures that the Curie point is reached for all particles and the target particle temperature of 44.5° C. is realized. The additional field strength from 20 to 25 kA/m does not cause significant temperature rise above the Curie temperature of 44.5° C. Subtracting 2.5° C. of heat loss and the target cancer cell temp of 42° C. is realized. Example Curie temperatures for selected nano-particle materials include: chromium bromide=37° C.; europium oxide=77° C. A mixture of these two materials, for example, would yield a new Curie temperature of 44.5° C., provided the right balance of chromium bromide and europium oxide is used to make a new mixed material particle.

### Arrhenius Curve for Low Temperature Hyperthermia

It is important to stay in the 42 to 42.25° C. temperature range or cooler as shown in FIG. 11, lines 1230, region 1240. Note the cell death rate is very small for this low temperature hyperthermia range. At 42° C., the probability of cell death almost flattens out and is relatively independent of time. In contrast, the cell death rate at 46.5° C. is almost vertical meaning cell death occurs almost instantaneously. Thus, in just a 4.5° C. span, the cell death rate goes from virtually zero to 100%. Thus, it is paramount that the cellular temperature be tightly controlled; and be targeted at 42° C. or less. Observe how dramatic the cell death rate is from 42.0° C. to 43.0° C. This underscores how important tight temperature control is and, correspondingly, how critical the particle design is in conjunction with the applied field strength. Being off by even as much as 1.0° C. causes this process to fail. Thus, designing the temperature control largely into the material properties of the particle is the critical inventive step necessary for success.

The Arrhenius curve is independent of whether the cells are in vivo (in the body) or in vitro (in the glass). Thermodynamic equations which describe the heat loss from the nano-particles, whether the particles are clumped in the cancer cell or whether the particles are evenly distributed in the cancer cell, enable the incorporation of heat loss to determine the optimal particle temperature. The physiological benefits of Low Temperature Hyperthermia, primarily the minimization of the release of cancer stem cells, require that the temperature range stay at 42° C. and cooler. Certain conditions affect the positioning of the Arrhenius curve and include acidification or step down hyperthermia and post thermal tolerance induction. These also need to be considered for a given patient treatment protocol.

### Benefits of Low Temperature Hyperthermia

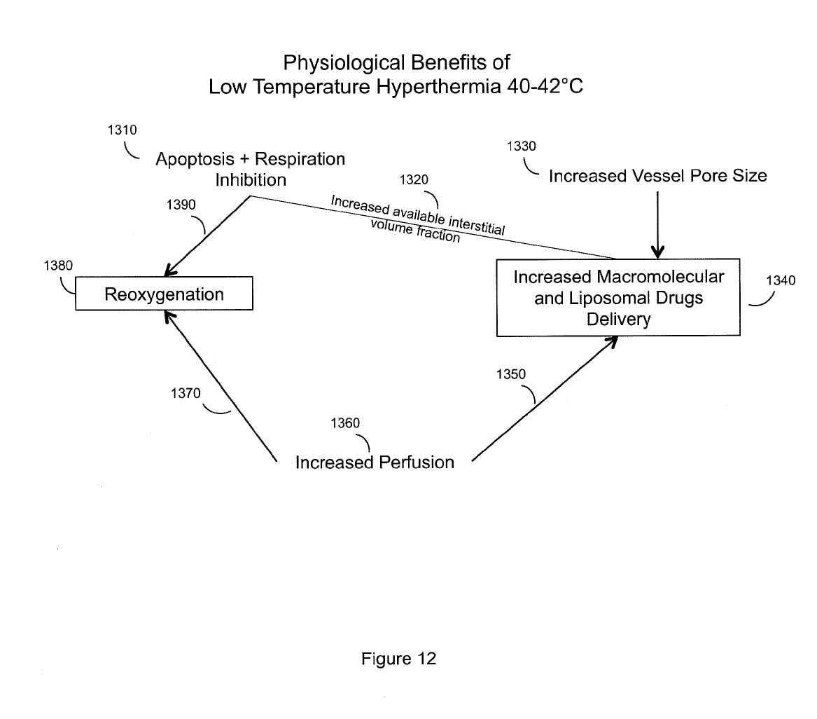

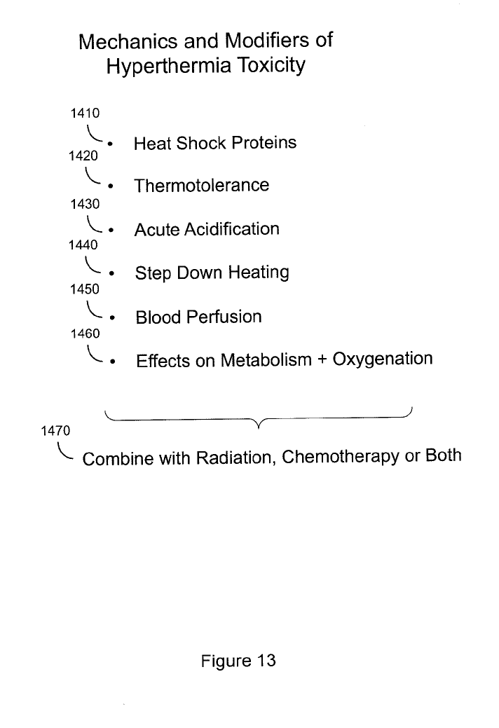

Some of the detailed benefits of Low Temperature Hyperthermia are shown in FIGS. 12 and 13. It has been suggested that these benefits are realized between the temperature range of 41° C. to 41.5° C. in skin. The optimal temperature is different for different tissue types and this description has used the target temperature of 42° C., but in practice this temperature could be anything that is optimal for a given tissue type.

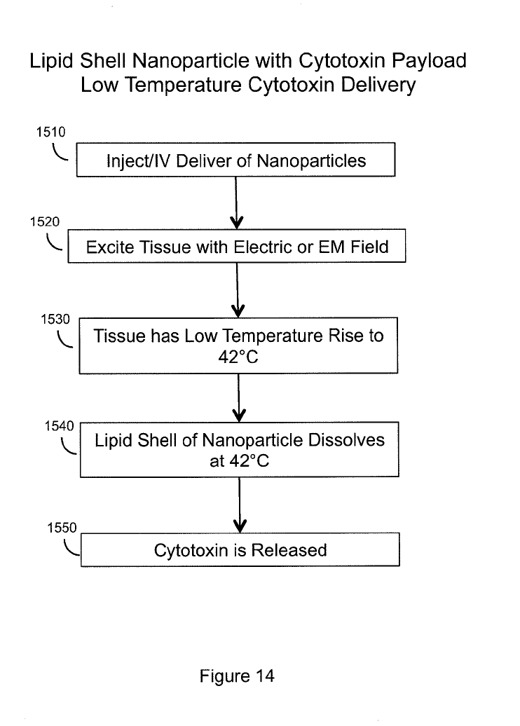

Of note, cancer cells can adapt to heat stress by becoming thermo-tolerant. This is caused by the release of Heat Shock Proteins. Thermo-tolerance tends to shift the Arrhenius curve down and to the right indicating higher temperatures are needed along with greater times at that temperature, to realize the same effect. Thus, minimizing the level of Heat Shock Proteins reduces the level of resistance to hyperthermia treatment. Low Temperature Hyperthermia has a number of beneficial effects: it improves Perfusion as shown at 1360, where skin perfusion can be 10-fold while tumor perfusion can be 1.5- to 2.0-fold. Increased blood vessel pore size is realized at 1330, where both of these effects improve drug delivery performance, such as via liposomes (lipid) as shown in FIG. 14. Increased profusion and blood vessel size also enhance re-oxygenation 1380, which is critical since cancer stem cells prefer a hypoxic environment. Thus, this helps kill cancer cells. In FIG. 12 at 1380 and FIG. 13 at 1460, enzymes for aerobic metabolism are more heat sensitive than those for anaerobic metabolism. Thus, during low temperature hyperthermia, there is a concomitant reduction in tumor respiration. Respiration inhibition is shown at 1310. Minimizing the level of Heat Shock Proteins is important, since cancer cells with Heat Shock Proteins are relatively resistant to hyperthermia treatment. In addition, at 1430, acute acidification of cancer cells below their resting pH leads to catastrophic cell death.

Step 1510 has the nano-particles delivered on site where said nano-particle is a lipid shell with a cytotoxin payload. Step 1520 excites the tissue with an external field, E or EM. The tissue slowly rises to 42° C. Alternatively, the delivery of a second set of nano-particles, those that are magneto-caloric or Electro-caloric or Curie sensitive, could bring the tissue temp to 42° C. In any event, when the tissue reaches 42° C. in this example, the blood vessel diameter is greater and the blood perfusion is greater, and the lipid shell layer dissolves away at step 1540 releasing the cytotoxin into the cancer cell at step 1550. This could be combined with a pre-treatment of radiation. This approach has the advantage of no cytotoxin being released either in the blood stream during transport to the cancer cells or into healthy cells, since they are not heated to 42° C. (should one of these cytotoxin nano-particles errantly reside in a healthy cell). This approach can use an electric field or EM-Field to cause a tissue temperature rise to 42° C. if no other method is available.

### SUMMARY

The Low Temperature Hyperthermia System uses specially designed nano-particles that exhibit a specific temperature rise in a given illumination energy field and then have no further temperature rise even if the applied illumination energy field increases beyond the optimal level. Alternatively, the nano-particles exhibit a tightly controlled temperature rise based on a pre-determined or pre-designed a priori temperature rise for a given illumination energy field strength. This ensures that an optimal treatment temperature is not exceeded in the tissue, which minimizes the release of Heat Shock Proteins, in addition to numerous physiological benefits, while further stressing the cancer cells so that they die, versus emitting cancer stem cells/other cells.

What is claimed as new and desired to be protected by Letters Patent of the United States is:

1. A method for treating invasive agents which are located in a living organism comprising:
implanting nano-particles inside of or proximate to an invasive agent which is located in a living organism;
generating an energy field which has a predetermined set of characteristics;
applying said energy field to said living organism to illuminate said nano-particles; and
raising a temperature of the invasive agent via the illumination of said nano-particles to a predetermined temperature.

2. The method of treating invasive agents of claim 1, further comprising:
treating said living organism with at least one of: chemotherapy, radiation, and release of a cytotoxin as at least one of a pre-treatment, post-treatment, and concurrent treatment in conjunction with raising a temperature of the invasive agent via the illumination of said nano-particles to a predetermined temperature.

3. The method of treating invasive agents of claim 1, further comprising:
dynamically controlling an intensity of the generated energy field to elevate a temperature of the invasive agent above an ambient temperature and maintain said temperature below a predetermined threshold.

4. The method of treating invasive agents of claim 1, further comprising:
dynamically controlling an intensity of the generated energy field to maintain a temperature of the invasive agent at a temperature elevated above an ambient temperature and below a predetermined threshold for a predetermined duration.

5. The method of treating invasive agents of claim 1, further comprising:
dynamically controlling an intensity of the generated energy field to elevate a temperature of the living organism in the vicinity of the invasive agent above an ambient temperature and maintain said temperature below a predetermined threshold.

6. The method of treating invasive agents of claim 1, further comprising:
dynamically controlling an intensity of the generated energy field to maintain a temperature of the living organism in the vicinity of the invasive agent at a temperature elevated above an ambient temperature and below a predetermined threshold for a predetermined duration.

7. The method of treating invasive agents of claim 1 wherein said step of implanting nano-particles comprises:
inserting nano-particles that exhibit a specific temperature rise in a given illumination energy field and then have no further temperature rise even if the applied illumination energy field increases beyond the optimal level.

8. The method of treating invasive agents of claim 1 wherein said step of implanting nano-particles comprises:
inserting nano-particles that exhibit a tightly controlled temperature rise based on a pre-determined or pre-designed a priori temperature rise for a given illumination energy field strength.

9. The method of treating invasive agents of claim 1 wherein said step of raising a temperature comprises:
raising a temperature of the invasive agent via the illumination of said nano-particles to approximately 42° C.

10. The method of treating invasive agents of claim 1 wherein the step of generating an energy field comprises:
controllably generating at least one of an electric field (E-Field), a magnetic field (H-Field), a combination of both an electric field (E-Field) and a magnetic field (H-Field), an optical field, and an acoustic field.

11. The method of treating invasive agents of claim 1 wherein said step of implanting nano-particles comprises:
inserting nano-particles inside of or proximate to a site in which an invasive agent resides via at least one of: intravenous delivery, in-situ injection, or topical application in said living organism.

12. A system for treating invasive agents which are located in a living organism wherein nano-particles are implanted inside of or proximate to an invasive agent which is located in a living organism, the system comprising:
an energy field generator for generating an energy field which has a predetermined set of characteristics;
energy radiating elements for applying said energy field to said living organism to illuminate said nano-particles; and
a controller for raising a temperature of the invasive agent via the illumination of said nano-particles to a predetermined temperature.

13. The system for treating invasive agents of claim 12, further comprising:
a treatment management process for treating said living organism with at least one of: chemotherapy, radiation, and release of a cytotoxin as at least one of a pre-treatment, post-treatment, and concurrent treatment in conjunction with raising a temperature of the invasive agent via the illumination of said nano-particles to a predetermined temperature.

14. The system for treating invasive agents of claim 12, further comprising:
an intensity controller for dynamically controlling an intensity of the generated energy field to elevate a temperature of the invasive agent above an ambient temperature and maintain said temperature below a predetermined threshold.

15. The system for treating invasive agents of claim 12, further comprising:
an intensity controller for dynamically controlling an intensity of the generated energy field to maintain a temperature of the invasive agent at a temperature elevated above an ambient temperature and below a predetermined threshold for a predetermined duration.

16. The system for treating invasive agents of claim 12, further comprising:
an intensity controller for dynamically controlling an intensity of the generated energy field to elevate a temperature of the living organism in the vicinity of the invasive agent above an ambient temperature and maintain said temperature below a predetermined threshold.

17. The system for treating invasive agents of claim 12, further comprising:
an intensity controller for dynamically controlling an intensity of the generated energy field to maintain a temperature of the living organism in the vicinity of the invasive agent at a temperature elevated above an ambient temperature and below a predetermined threshold for a predetermined duration.

18. The system for treating invasive agents of claim 12 wherein said nano-particles exhibit a specific temperature rise in a given illumination energy field and then have no further temperature rise even if the applied illumination energy field increases beyond the optimal level.

19. The system for treating invasive agents of claim 12 wherein said nano-particles exhibit a tightly controlled temperature rise based on a pre-determined or pre-designed a priori temperature rise for a given illumination energy field strength.

20. The system for treating invasive agents of claim 12 wherein said controller comprises:
an illumination manager for raising a temperature of the invasive agent via the illumination of said nano-particles to approximately 42° C.

21. The system for treating invasive agents of claim 12 wherein the energy field generator comprises:
a generator controller for controllably generating at least one of an electric field (E-Field), a magnetic field (H-Field), a combination of both an electric field (E-Field) and a magnetic field (H-Field), an optical field, and an acoustic field.

22. The system for treating invasive agents of claim 12 wherein said nano-particles are inserted inside of or proximate to a site in which an invasive agent resides via at least one of: intravenous delivery, in-situ injection, or topical application in said living organism.

23. A method for treating invasive agents which are located in a living organism comprising:
implanting nano-particles inside of or proximate to an invasive agent which is located in a living organism;
generating an energy field which has a predetermined set of characteristics;
applying said energy field to said living organism to illuminate said nano-particles;
raising a temperature of the invasive agent via the illumination of said nano-particles; and
dynamically controlling an intensity of the generated energy field to maintain a temperature of at least one of the invasive agent and the living organism above an ambient temperature and below a predetermined threshold.

24. The method of treating invasive agents of claim 23, further comprising:
treating said living organism with at least one of: chemotherapy, radiation, and release of a cytotoxin as at least one of a pre-treatment, post-treatment, and concurrent treatment in conjunction with raising a temperature of at least one of the invasive agent and the living organism via the illumination of said nano-particles to a predetermined temperature.

25. The method of treating invasive agents of claim 23 wherein said step of implanting nano-particles comprises:
inserting nano-particles that exhibit a specific temperature rise in a given illumination energy field and then have no further temperature rise even if the applied illumination energy field increases beyond the optimal level.

26. The method of treating invasive agents of claim 23 wherein said step of implanting nano-particles comprises:
inserting nano-particles that exhibit a tightly controlled temperature rise based on a pre-determined or pre-designed a priori temperature rise for a given illumination energy field strength.

27. The method of treating invasive agents of claim 23 wherein said step of raising a temperature comprises:
raising a temperature of at least one of the invasive agent and the living organism via the illumination of said nano-particles to approximately 42° C.

28. The method of treating invasive agents of claim 23 wherein said step of implanting nano-particles comprises:
inserting nano-particles inside of or proximate to a site in which an invasive agent resides via at least one of: intravenous delivery, in-situ injection, or topical application in said living organism.
